(19) United States
(12) Patent Application Publication
(10) Pub. No.: US 2026/0285677 A1
(43) Pub. Date: Sep. 24, 2026

(54) A METHOD FOR STORING HYDROGEN IN A REACTOR OR A SYNTHESIS LOOP

(71) Applicant: Topsoe A/S, Kgs. Lyngby (DK)

(72) Inventors: Per Aggerholm Sørensen, Kgs.Lyngby (DK); Ameet Kakoti, Kokkedal (DK); Anders Berthelsen, Kgs.Lyngby (DK)

(73) Assignee: Topsoe A/S, Kgs. Lyngby (DK)

(21) Appl. No.: 19/474,621

(22) PCT Filed: Apr. 22, 2024
(86) PCT No.: PCT/EP2024/060908

§ 371 Date: Oct. 10, 2025

(30) Foreign Application Priority Data
Apr. 27, 2023 (DK) PA202330004

(51) Int. Cl. C01B 3/025; C01C 1/04; C07C 29/151; C07C 29/152; C07C 29/76

(52) U.S. Cl. CPC C01B 3/025; C01C 1/04; C07C 29/1516; C07C 29/152; C07C 29/76

(57) ABSTRACT

A method for storing hydrogen in a reactor or a synthesis loop comprising the steps of (a) providing a gaseous stream of a reaction compound; (b) providing an excess of a hydrogen stream as required for stoichiometric molar ratio of reactants to hydrogen in the synthesis loop or reactor from an electrolysis unit; (c) storing the excess of hydrogen provided in step (b) by introducing at least an amount of the hydrogen stream into the gaseous stream of a reaction compound and to provide a mixed stream of hydrogen and gaseous reaction compound with at least 25 mol % excess hydrogen than what is required for a reaction of the reaction compound with hydrogen in the hydrogen stream; (d) introducing the mixed stream into the reactor or the synthesis loop; (e) withdrawing a mixed stream of gaseous reaction product and unreacted gaseous hydrogen and reaction compound from the reactor or the synthesis loop; (f) separating the reaction product from the unreacted gaseous hydrogen and reaction compound (g) recycling all or a part of unreacted amounts of hydrogen and reaction compound to the reactor or synthesis loop.

The present invention is directed towards a method of storing excess of green hydrogen in a reactor or synthesis loop in processes where hydrogen is produced by electrolysis of water and wherein the electrolysis is operated on power from renewable energy to prepare the so-called green hydrogen.

Green hydrogen is a type of hydrogen that is produced using renewable energy sources, such as solar, wind, or hydropower. Unlike traditional hydrogen production methods, which rely on fossil fuels, green hydrogen is produced by using electrolysis to split water into hydrogen and oxygen.

Green hydrogen has gained attention in recent years as a potential solution for decarbonizing industries such as transportation, energy, and manufacturing. It can be used as a fuel for vehicles, a source of energy for power plants, and a feedstock for industrial processes.

The energy needed for electrolysis is generated from renewable sources, making the production of green hydrogen a sustainable and low-carbon alternative to traditional hydrogen production methods.

A problem with utilizing green technology based on renewable energy for production of chemicals is the energy is fluctuating. This means that the load on the plant will vary depending on the available hydrogen and/or available reaction compounds, typically delivered from external sources. Excessive production of hydrogen will typically be in situations where supply of renewable energy periodically is higher than what is needed for the consumption of the hydrogen by reaction with a reactant in the reactor or the synthesis loop.

Water electrolysis can be affected by a number of different factors that can cause fluctuations in its performance. Some of the key factors that can affect water electrolysis:
1. Voltage fluctuations: Electrolysis requires a constant voltage to be applied across the electrodes to split water molecules into hydrogen and oxygen. Any fluctuations in the voltage can affect the efficiency of the process and result in inconsistent hydrogen production.
2. Temperature variations: Electrolysis is temperature-dependent, and changes in temperature can affect the conductivity of the electrolyte and the performance of the electrodes. High temperatures can also cause thermal degradation of the electrodes, reducing their lifespan.

Thus, one of the challenges with green technology is the insufficient supply of reactants in the production of green hydrogen through water electrolysis, the supply of renewable electricity can be limited, which can result in insufficient hydrogen production. Similarly, the availability of feedstocks, such as carbon oxides or nitrogen, for reaction with hydrogen, can be limited.

An approach to addressing the insufficient supply of reactants is to develop closed-loop systems that recycle and reuse reactants. For example, in the production of methanol and ammonia, the unconverted reactants are recycled back into the process to reduce the need for additional feedstocks. In the case of green hydrogen production, excess hydrogen can be stored and reused to offset fluctuations in the supply of renewable electricity.

Alternatively, excess of hydrogen may exist when supply of reactants is in deficit to what is required for the reaction with hydrogen.

This can be solved to utilizing an intermediate storage for hydrogen where excess hydrogen is stored during periods with excess hydrogen production. This hydrogen will be released during periods with insufficient production of hydrogen to maintain a satisfactory production level.

A separate hydrogen storage is very costly. Hydrogen storage refers to the methods and technologies used to store hydrogen for use as a fuel or for other purposes. Hydrogen is a lightweight, high-energy fuel that can be used in fuel cells to generate electricity, or in combustion engines to power vehicles. However, hydrogen has a low energy density, which means that it requires large storage volumes to hold enough fuel for practical applications.

There are several methods of storing hydrogen, including:
1. Compressed hydrogen storage: This method involves compressing hydrogen gas to high pressures (typically 350-700 bar) and storing it in high-pressure tanks or pipelines. Compressed hydrogen storage is relatively simple and costeffective, but it requires heavy and bulky storage tanks.
2. Liquid hydrogen storage: Hydrogen can also be stored in a liquid state by cooling it to −253° C., at which point it becomes a liquid. Liquid hydrogen storage offers higher energy density than compressed hydrogen storage, but it requires even more extreme cooling and specialized equipment.
3. Metal hydride storage: Metal hydrides are materials that can absorb and release hydrogen gas under certain conditions. These materials offer a high storage capacity and can be compact and lightweight, but they can be expensive and may have limited durability.

Each of these methods has advantages and disadvantages depending on the specific application and requirements.

In some cases, the unreacted reactants are recycled directly back into the reactor without any treatment. In other cases, the unreacted reactants may be separated from the product and purified before being recycled back into the reactor.

The recycle of unconverted reactants is commonly used in large-scale chemical processes, such as the production of ammonia, methanol, and other chemicals. It allows for the efficient use of resources and can help to reduce the environmental impact of chemical production.

Overall, the recycling of unconverted reactants to a reactor is a valuable strategy in chemical engineering that can help to increase the efficiency and yield of chemical reactions. However, careful design and management are necessary to ensure that the system operates safely and effectively.

Overall, addressing the insufficient supply of reactants in green technology requires a multi-faceted approach that involves developing new methods for producing renewable energy and finding alternative sources of feedstocks, as well as implementing closed-loop systems that recycle and reuse materials.

Using fluctuating sustainable energy in the electrolysis will periodically result in either excess or deficit of hydrogen. Thus, it will be necessary to store excess of hydrogen and use the stored hydrogen, in periods of time with deficit of hydrogen.

The invention is based on utilizing an existing synthesis loop as temporary hydrogen storage, e.g., capacity for 15-30 minutes of operation in order to avoid an expensive separate hydrogen storage. This can be done by allowing storage of hydrogen in the synthesis loop in an amount higher than the normal stoichiometric composition of the synthesis gas as mentioned hereinbefore of (H2-CO2)/(CO+CO2) for the methanol synthesis or H2/N2 for the ammonia synthesis when increasing the hydrogen concentration compared with normal conditions.

As mentioned hereinbefore and in the following description and claims, the stoichiometric composition refers the synthesis gas composition before the synthesis gas is mixing with the unreacted reactants, i.e., the composition of the so-called make-up gas. Due to a high tolerable pressure in the reactor or the synthesis loop a decent amount of hydrogen can be stored.

A methanol loop is normally operated at 80-90 barg. Since the methanol synthesis already contains a high-pressure volume (loop) and make-up compressor it will be simpler and less costly to design these components for higher pressure instead of separate hydrogens storage facility.

The pressure increase in the reactor or the synthesis loop varies depending on the amount of hydrogen added into the reactor or loop to be stored. Correspondingly, the pressure in the reactor or the synthesis loop is reduced to normal again, when excess of hydrogen is consumed.

The present invention provides a method for storing hydrogen in a reactor or a synthesis loop comprising the steps of
(a) providing a gaseous stream of a reaction compound;
(b) providing an excess of a hydrogen stream as required for stoichiometric molar ratio of reactants to hydrogen in the synthesis loop or reactor from an electrolysis unit;
(c) storing the excess of hydrogen provided in step (b) by introducing at least an amount of the hydrogen stream into the gaseous stream of a reaction compound and to provide a mixed stream of hydrogen and gaseous reaction compound with at least mol % excess hydrogen than what is required for a reaction of the reaction compound with hydrogen in the hydrogen stream;
(d) introducing the mixed stream into the reactor or the synthesis loop;
(e) withdrawing a mixed stream of gaseous reaction product and unreacted gaseous hydrogen and reaction compound from the reactor or the synthesis loop;
(f) separating the reaction product from the unreacted gaseous hydrogen and reaction compound
(g) recycling all or a part of unreacted amounts of hydrogen and reaction compound to the reactor or synthesis loop.

In a synthesis loop, reactants and hydrogen are continuously introduced into the reactor vessel, where they undergo a chemical reaction. The resulting products are then withdrawn from the reactor and unconverted gaseous reactants and hydrogen are separated from the reaction product and then returned to the reactor vessel to continue the reaction cycle.

Synthesis loops are used in a variety of chemical, including the production of methanol, ammonia and fuels.

In a specific embodiment of the invention, the gaseous reaction compound is carbon dioxide or carbon monoxide or a mixture thereof for use in the methanol synthesis.

The methanol synthesis is usually performed in a reactor loop.

A synthesis loop is a closed-loop system beside a purge gas stream typically required to avoid buildup of inert compounds.

The loop is used for the continuous production of methanol through a chemical reaction between carbon monoxide and/or carbon dioxide, and hydrogen. It typically consists of a series of reactors, heat exchangers, and separation units.

In the methanol synthesis loop, a mixture of carbon monoxide and/or carbon dioxide, and hydrogen is continuously fed into one or multiple reactors, where it undergoes a chemical reaction to methanol.

After leaving the reactor, the reaction mixture is cooled and fed into a separation system, where the methanol is separated from the unreacted gases and impurities. The unreacted gases are recycled back into the synthesis loop to continue the reaction cycle, while the purified methanol is collected for further processing and use.

Methanol synthesis gas preferably has a composition corresponding to a so-called module (M=(H2-CO2)/(CO+CO2)) of 1.90-2.20.

When excess of hydrogen must be stored intermediately in the synthesis loop, module M=(H₂—CO₂)/(CO+CO₂) will be typically higher than 2.5, such as higher than 3 or higher than 3.5 depending on the amount of stored hydrogen.

In further a specific embodiment of the invention, the gaseous reaction compound is nitrogen for use in the ammonia synthesis in an ammonia loop.

An ammonia synthesis loop is a closed-loop system used for the continuous production of ammonia through the reaction of nitrogen with hydrogen.

In the process, nitrogen and hydrogen gases are fed into a reactor at high pressure and temperature, where they react to form ammonia gas.

After leaving the reactor, the reaction mixture is cooled and fed into a separation system, where the ammonia gas is separated from the unreacted nitrogen and hydrogen gases. The unreacted gases are recycled back into the synthesis loop to continue the reaction cycle, while the purified ammonia gas is collected for further processing and use.

In the ammonia synthesis gas, the molar ratio, i.e. module of hydrogen to nitrogen is preferably 3:1.

In a specific embodiment of the invention, if excess of hydrogen must be stored in the ammonia loop, the hydrogen/nitrogen molar ratio will be periodically higher than 3.75, such as higher than 4.5 or higher than 5.

In another specific embodiment of the invention, the gaseous reaction compound is pure carbon monoxide, which will typically be used in the Fischer-Tropsch reaction The Fischer-Tropsch reaction is a chemical process used to convert carbon monoxide (CO) and hydrogen (H2) gases into liquid hydrocarbons, such as gasoline, diesel, and other synthetic fuels.

The reaction involves catalytic reaction of H2 and CO gases in a molar ratio of 2. In periods with excess production of hydrogen, hydrogen is according to the invention stored in the reactor and the molar ratio of H2 to CO will be higher than 2.5, such as higher than 3 or higher than 3.5.

In all of the above embodiments of the invention, the synthesis gas composition is adjusted to lower than the normal molar ratio of hydrogen to gaseous reactants, e.g. CO2 when the period with excess hydrogen production is completed. This occurs to consume the excess of hydrogen in the reactor or synthesis loop by reaction with the reactants.

### EXAMPLE

As an example of the invention below is shown the additional hydrogen storage capacity for a 150 MTPD methanol loop normally operating at 80 bar g when pressurized to 100 bar g. The methanol loop system gas volume is around 55 m³ at an average temperature of 220° C. for the capacity of 150 MTPD

| | Case A | Case B |
|---|---|---|
| Loop pressure (bar g) | 80 | 120 |
| Stored excess hydrogen | 0 | 1200 |
| (Nm3) | | |

As the corresponding hydrogen consumption for 150 MTPD methanol production is approximately 13,000 Nm3/h the loop contains excess hydrogen for 5 minutes of operation.

1. A method for storing hydrogen in a reactor or a synthesis loop comprising the steps of
(a) providing a gaseous stream of a reaction compound;
(b) providing an excess of a hydrogen stream as required for stoichiometric molar ratio of reactants to hydrogen in the synthesis loop or reactor from an electrolysis unit;
(c) storing the excess of hydrogen provided in step (b) by introducing at least an amount of the hydrogen stream into the gaseous stream of a reaction compound and to provide a mixed stream of hydrogen and gaseous reaction compound with at least 25 mol % excess hydrogen than what is required for a reaction of the reaction compound with hydrogen in the hydrogen stream;
(d) introducing the mixed stream into the reactor or the synthesis loop;
(e) withdrawing a mixed stream of gaseous reaction product and unreacted gaseous hydrogen and reaction compound from the reactor or the synthesis loop;
(f) separating the reaction product from the unreacted gaseous hydrogen and reaction compound
(g) recycling all or a part of unreacted amounts of hydrogen and reaction compound to the reactor or synthesis loop.

2. The method of claim 1, wherein the gaseous reaction compound is carbon dioxide or carbon monoxide or a mixture thereof.

3. The method of claim 2, wherein the reaction is methanol synthesis.

4. The method of claim 3, wherein module M=(H₂—CO₂)/(CO+CO₂) is higher than 2.5, preferably higher than 3.

5. The method of claim 3, wherein module M=(H₂—CO₂)/(CO+CO₂) is higher than 3.5

6. The method of claim 1, wherein the gaseous reaction compound is nitrogen.

7. The method of claim 6, wherein the reaction is ammonia synthesis.

8. The method of claim 7, wherein the molar ratio of H2/N2 is higher than 3.75, preferably higher than 4.5.

9. The method of claim 7, wherein the molar ratio of H2/N2 is higher than 5.

10. The method of claim 1, wherein the gaseous reaction compound is carbon monoxide.

11. The method of claim 10, wherein the reaction is a Fischer-Tropsch reaction.

12. The method of claim 11, wherein the H2/CO molar ratio is higher than 2.5, preferably higher than 3.

13. The method of claim 11, wherein the H2/CO molar ratio is higher than 3.5.
